(19) United States
(12) Patent Application Publication
(10) Pub. No.: US 2010/0222568 A1
(43) Pub. Date: Sep. 2, 2010

(54) Removal of pyridine and pyridine analogs from reaction mass containing sucrose esters

(76) Inventors: Rakesh Ratnam, Banglore (IN); Sundeep Aurora, Banglore (IN)

Correspondence Address: THE NATH LAW GROUP, 112 South West Street, Alexandria, VA, 22314, US

(21) Appl. No.: 11/991,169

(22) PCT Filed: Aug. 29, 2006
(86) PCT No.: PCT/IN2006/000326

§ 371 Date: Feb. 28, 2008

(30) Foreign Application Priority Data
Aug. 30, 2005 (IN) 1045/MUM/2005

(51) Int. Cl. C07H 11/00; C07H 1/06

(52) U.S. Cl. 536/115; 536/127

(57) ABSTRACT

A process of removal of pyridine or a pyridine analogue from a composition or a Process Stream in a process of production of 4,1′, 6′ trichlorogalactosucrose is described comprising reacting pyridine with an acid, the said acid being used preferably in gaseous form, achieving complete precipitation of the salt of pyridine in higher alcoholic solvents and non-polar solvents, filtering off the precipitate of the said salt of pyridine to achieve removal of pyridine from the reaction system and optionally regenerating and recovering pyridine by reacting the said salt with alkali.

### TECHNICAL FIELD

The present invention relates to a process and a novel strategy for production, isolation and purification of sucrose-6-ester, which ultimately is used as starting material in production of 1′-6′-Dichloro-1′-6′-DIDEOXY-β-Fructofuranasyl-4-chloro-4-deoxy-galactopyranoside and other chlorinated sucrose compounds.

### BACKGROUND OF INVENTION

Chlorinated sucrose preparation is a challenging process due to the need of chlorination in selective less reactive positions in sucrose molecule in competition with more reactive positions. Generally, this objective is achieved by a procedure which involves essentially protecting the hydroxy group in the pyranose ring of sugar molecule by using various protecting agents such as alky/aryl anhydride, acid chlorides, orthoesters etc., and the protected sucrose is then chlorinated in the desired positions (1′-6′ &, 4) to give the acetyl derivative of the product, which is then deacylated to give the desired product 1′-6′-Dichloro-1′-6′-DIDEOXY-β-Fructofuranasyl-4-chloro-4-deoxy-galactopyranoside i.e. 4,1′, 6′ trichlorogalactosucrose (TGS). However, in these methods, substitutions at undesired position cannot be totally avoided and such products get mixed as impurities. Regio-selective substitution at desired position is possible by Regio-selective reactions either by using soluble or immobilized tin containing catalysts.

Sucrose-6-esters can also be produced as a major product by reacting sucrose and an acylating agent in the presence of pyridine analogs, picolines etc. under low temperature conditions. However, after the esterification reaction, the complete removal of pyridine and such compounds poses a major process constraint. This invention is related to the complete removal of pyridine analogs after such esterification reaction. Further purification of the sucrose esters becomes easier after the removal of the said analogs.

Thus, Sucrose-6-ester is produced by direct acetylation or benzoylation of sucrose dissolved in pyridine analog compounds. This reaction is carried out at temperature below −20° C. to −40° C. After the formation of the sucrose-6-ester, the reaction mixture containing the said ester is purified and taken for the chlorination reaction using Vilsmeier reagent.

The purification of sucrose-6-ester from the above process poses a major process constraint due to the presence of pyridine or such compounds as aromatic nitrogenous bases such as picoline, pyrrolidine, etc. They are removed conventionally by distillation. However, pyridine and its analogues are high boiling solvents too. They need to be removed under reduced pressure and they are rarely removed completely from the reaction mixture by distillation under reduced pressure. Further, handling of pyridine in distillation process is also a major bottleneck when the process is scaled up to industrial scale. The maximum permissible standards for exposure of human beings to pyridine or its analogs are very stringent. The present international standards allow the Permitted Daily Exposure (PDE) at a very low level of less than 3 mg/day. Still further, the residual solvent, pyridine and its analogs, allowed is less than 200 ppm. Hence an effective removal of pyridine or its analogs to a better extent than is possible presently is an absolute need.

### PRIOR ART

Mufti et al (1983) (U.S. Pat. No. 4,380,476) have reported the conventional process of acylation in which sucrose is reacted with pyridine and acetic anhydride at a temperature of −20degree to −70degree. C. To the above reaction mixture, which still contains pyridine, chloroform was added and the contents cooled to −75.degree. C. in a dry ice/acetone bath. The chloroform was added primarily to prevent freezing of pyridine but also to slow down the reaction and thus allow better control over the reaction. Sulphuryl chloride was then added to the cooled reaction mixture dropwise over a period of 1.5 hours. The reaction mixture was then allowed to warm to room temperature and left at that temperature for 4 hours, after which time it was heated at 45.degree. C. for 12 hours and then cooled to room temperature. The mixture was poured into pre-cooled (about 4.degree. C.) 10% sulphuric acid solution (100 ml) slowly with stirring. The sulphuric acid mixture was extracted twice with chloroform and the chloroform extracts washed twice with water, with saturated sodium hydrogen carbonate solution pH 7 and then twice with water, and dried over anhydrous sodium sulphate. Pyridine got removed in the saturated sodium hydrogen carbonate washings given to chloroform extract. Further removal in water washings to chloroform extract.

Besides the conventional process of chlorination as described above, pyridine is also used for various other process steps in the production of TGS.

Thus tritylation of sucrose to block the three primary alcohol groups is accomplished by reacting sucrose with trityl chloride in a suitable solvent such as pyridine (U.S. Pat. No. 4,783,526). If pyridine is used as a solvent, the same is removed by pouring the reaction mixture after acetylation into ice water and the precipitated product filtered and dried and the procedure is repeated a number of times to remove any traces of pyridine. Pyridine is also used in acetyl migration step of 2,3,4,3′,4′-penta-O-acetyl sucrose. Process of preparation of TGS from Tetrachlororaffinose also involves use of pyridine as a solvent. U.S. Pat. No. 4,889,928 has described use of pyridine and containing 4 to 8 molar equivalents of water and toluene p-sulphonic acid or hydrochloric acid having a pH of about 5 to 6 for providing conditions for subjecting a sucrose alkyl 4,6-orthoacylate to mild aqueous acidic hydrolysis. U.S. Pat. No. 4,977,254 described use of pyridine for reaction of sugar or partly protected sugar with thionyl chloride. U.S. Pat. No. 5,449,772 has described use of pyridine as one of the inter solvents for reacting a solution of sucrose with a reagent selected from the group consisting of a trialkyl orthoester and a ketene acetal, in the presence of an acid catalyst to provide a sucrose alkyl 4,6-orthoester, U.S. Pat. Nos. 6,998,480 and 7,049,435 have mentioned use of pyridine as one of the solvents that can be used in a solvent extraction approach.

### SUMMARY OF THE INVENTION

Invention as described here involves removal of pyridine from a reaction mixture or a Process Stream by reacting the same with an acid, removing water from the reaction mixture/Process Stream to ensure complete precipitation of the salt of pyridine, filtering off the precipitate to achieve removal of pyridine from the reaction system. If pyridine is required to be removed in large quantities, it is preferably removed as much as possible by distillation under reduced pressure. Rest of the pyridine remaining in the reaction mixture is removed by reacting with acid to form a salt, as mentioned before.

The pyridine salt can be reacted with alkali to regenerate and recover pyridine for re-use.

### DETAILED DESCRIPTION OF THE INVENTION

Preferred embodiment of this invention is removal of pyridine or its analogues from esterification of sucrose by an esterifying agent in presence of pyridine.

After Sucrose-6-ester has been produced as a major product by reacting sucrose and an acylating agent in the presence of pyridine, pyridine analogs including picolines etc. under low temperature conditions, the water in the system is completely removed by azeotropic distillation using cyclohexane and the pyridine from the reaction mixture is removed up to 50-60% of its initial volume by distillation. Then an equal amount of an alcoholic solvent such as isopropanol, t-butanol etc., is replenished to the reaction mixture. Dry Hydrogen chloride gas is then purged into the reaction mixture for several hours slowly at 0 to −10° C. till pH of the reaction mass was less than 3.0. Pyridine or its analogs with dry HCl gas form the respective hydrochlorides, which precipitate out of the reaction mass in solid form. When the pyridine in the reaction mass is completely converted to pyridinium hydrochloride, the mass is then filtered under nitrogen to remove the said compound.

The filtrate containing the sucrose-6-ester dissolved in the appropriate alcoholic solvent is practically free from pyridine or its analogues, much below the maximum permissible level of 0.1% of residual pyridine and its analogs and can be taken for further purification after subsequent removal of the alcoholic solvent.

The ester group can be acetyl or benzoyl. HCl may also be replaced by other acid if it could be safely handled. Further, the concept of pyridine removal by converting it into its hydrochloride form will work for any of the other processes of production of TGS where pyridine is used for purposes other than for facilitating acetylation. However the precipitation is facilitated only when the mass is taken into higher alcoholic solvents or nonpolar solventsProcess Stream to which this approach of pyridine removal can be applied may also be related to a process other than acetylation for synthesis of TGS or TGS-precursor including, but not limited to, tritylation of sucrose (U.S. Pat. No. 4,783,526), process of preparation of TGS from Tetrachlororaffinose, subjecting a sucrose alkyl 4,6-orthoacylate to mild aqueous acidic hydrolysis (U.S. Pat. No. 4,889,928), use of pyridine for reaction of sugar or partly protected sugar with thionyl chloride (U.S. Pat. No. 4,977,254), use of pyridine as one of the inert solvents for reacting a solution of sucrose with a reagent selected from the group consisting of a trialkyl orthoester and a ketene acetal in the presence of an acid catalyst to provide a sucrose alkyl 4,6-orthoester (U.S. Pat. No. 5,449,772), use of pyridine as one of the solvents that can be used in a solvent extraction approach (U.S. Pat. No. 6,998,480 and U.S. Pat. No. 7,049,435) and the like.

The examples given below are only illustrations of preferred embodiment of this invention. They shall in no way be considered to lessen the scope of the invention with respect to actual chemicals used, actual reaction conditions used and the like. Any adaptation or modification of the embodiments described here or new embodiments that are within the scope of the claims which are obvious to a person skilled in the art are considered as within the scope of this specification. Similarly, any mention of singular is also meant to cover its pleural also unless the context does not permit so. Thus, “an acid” covers use of all known acids which can be used for the purpose indicated therein. Similarly, a generic mention shall cover all the specific members of that kind. Thus “Esterification” covers acetylation, benzoylation and the like. “A pyridine analogue” covers one or more of and every analogue of pyridine comprising α-picoline, pyrrolidine and the like.

Further, even when not mentioned explicitly, mention of “Pyridine” includes mention of Pyridine analogues too, unless the context does not permit so.

### Example 1

### Precipitation of Pyridine Hydrochloride in Isopropanol

20 kg of sucrose was dissolved in 200 L of pyridine at 115° C. under reflux. After complete dissolution, the mixture was cooled to room temperature and further cooled to −30° C. 9.0 L of acetic anhydride was added dropwise to carry out Acetylation. The temperature was maintained between −30 and −35° C. with constant stirring. The formation of sucrose-6-acetate was monitored by TLC.

At the end of 4-5 hours, the reaction was terminated by addition of 2 L of water. Then the water was removed azeotropically using cyclohexane. Then the reaction mass was subjected to vacuum distillation where 112 L of pyridine was recovered. The reaction mass was then replenished with 112 L of isopropanol and chilled to −7° C.

Dry HCl gas was purged into the reaction mass till the pH reached 2.5-3.0. The formation of Pyridinium hydrochloride was indicated by solids precipitations. The mixture was held at −10° C. for 5-6 hours and then filtered through the nutsch filter.

The filtrate was analyzed for pyridine content and was found to be less than 0.1%, which is far less than the pyridine removal that is possible otherwise than the method of this invention.

The isopropanol was evaporated off and a thick mass of sucrose-6-acetate was obtained. It was seen that the thick mass contained unreacted sugar up to the maximum level of 2 percent of the mass and the 6-acetyl sucrose obtained was 72%

### Example 2

### Precipitation of Picoline Hydrochloride in t-Butanol

500 g of sucrose was dissolved in 4 L of α-picoline at 100° C. After complete dissolution, the mixture was cooled to room temperature and further cooled to −34° C. 360 g of benzoic anhydride was dissolved in 1.5 L of DMF and was added dropwise to carry out benzoylation. The temperature was maintained between −30 and −35° C. with constant stirring. The formation of 6-O-benzoyl sucrose was monitored by TLC.

At the end of 7-8 hours, the reaction was terminated by addition of 50 ml of water. Then the water was removed azeotropically using cyclohexane. Then the reaction mass was subjected to vacuum distillation where 1.8 L of α-picoline was recovered. The reaction mass was then replenished with 1.8 L of t-butanol and chilled to −12° C.

Dry HCl gas was purged into the reaction mass till the pH reached 2.5-3.0. The formation of α-picoline hydrochloride was indicated by solids precipitations. The mixture was held at −10° C. for 5-6 hours and then filtered through the nutsche filter.

The filtrate was analyzed for α-picoline content and was found to be less than 0.05%

The t-butanol was evaporated off and a thick mass of sucrose-6-benzoate was obtained. It was seen that the thick mass contained unreacted sugar up to the maximum level of 2 percent of the mass.

### Example 3

### Recovery of Pyridine from Pyridine Hydrochloride

The pyridine hydrochloride formed from Example 1 (120 kg) was suspended in 360 L of DM water and stirred thoroughly. Sodium hydroxide solution was added and the pH was adjusted to 9.0. The solution was then stirred for 60 minutes. The pyridine formed was fractionated through conventional distillation system. The pyridine recovered from the input for the batch was 90%.

The same process can be followed to recover α-picoline from α-picoline hydrochloride.

### Example 4

### Chlorination of Sucrose-6-acetate

31.5 kg of PCl₅ was added to 60 kg of DMF at room temperature and the Vilsmeier reagent was allowed to form. The POCl₃ generated in situ reacts with excess of DMF present and forms the second Vilsmeier. Both the Vilsmeier was mixed thoroughly and then cooled to 0° C.

10 kg of sucrose-6-acetate equivalent was dissolved in 30 L of DMF and was added to the reaction mass drop wise under stirring. After the complete addition of the 6-acetyl sucrose solution, the reaction mass was stirred for 30 minutes and was allowed to attain ambient and then further stirred for 60 minutes.

Then the reaction mixture was heated to 85° C. and was maintained for 60 minutes. The reaction mixture was then heated to 100° C. and maintained for 6 hours and then further heated to 115° C. and maintained for 2 hours.

The chlorinated reaction mass was then neutralized using calcium hydroxide slurry in water and the pH was adjusted to 7.0. The formation of TGS was analyzed by HPLC and the overall yield obtained was 40%.

### Example 5

### Removal of Pyridine from Trityl Chloride Reaction

10 kg of sucrose was dissolved in 60 L of pyridine at 70° C. 27.0 kg of Trityl chloride was added to the reaction flask and heated to 65° C. and maintained for 16 hrs. Then the reaction mass was cooled to 25-30° C. 6.0 kg of Acetic anhydride was added and stirred for 13-14 hrs for acetylation. 32 L of pyridine was removed by distilling under vacuum at 55° C.

t-butanol was added three times in volume to the reaction mass and HCl gas was purged for the conversion of pyridine to its hydrochloride. The precipitate started forming slowly and mass was kept stirring for 5 hours. The precipitate was then filtered through a nutsche filter and the filtrate was subjected to distillation under vacuum at 55-60° C. The solids then precipitated as the t-butanol concentration decreased in the filtrate and the solids were taken for further processing for the manufacture of TGS.

1. A process of removal of pyridine or a pyridine analogue from a composition or a Process Stream from a process of production of a precursor of 4,1′, 6′ trichlorogalactosucrose (TGS) comprising steps of:
a. reacting pyridine in a Process Stream from a reaction mixture by reacting the same with an acid,
b. achieving complete precipitation of the salt of pyridine in higher alcoholic solvents and non-polar solvents,
c. filtering off the precipitate of the said salt of pyridine to achieve removal of pyridine from the reaction system, and
d. optionally regenerating and recovering pyridine by reacting the said salt with alkali.

2. A process of claim 1 wherein prior to treatment with an acid, bulk of the pyridine or pyridine analogue from the Process Stream is removed by distillation, preferably under reduced pressure.

3. A process of claim 1 comprising:
a. use of hydrochloric acid as the preferred acid used for reacting with pyridine or pyridine analogues contained in the said Process Stream,
b. which, further preferably is in a dry gaseous form,
c. preferably purged into the reaction mass till the pH reached 2.5-3.0 leading to formation of Pyridinium hydrochloride indicated by solids precipitations,
d. holding the mixture at −10° C. for 5-6 hours and
e. then filtering through a filter, preferably a nutsche filter.

4. A process of claim 1 comprising reacting sucrose with a tritylating agent and acetylating the tritylated reaction product with an acetylating agent to obtain 6,1′,6′-tri-O-tritylsucrose penta-acetate.

5. A process of claim 1 for preparation of a sucrose 6-acylate which comprises subjecting a sucrose alkyl 4,6-orthoacylate to mild aqueous acidic hydrolysis by using pyridine as a reaction medium to provide a mixture of 4- and 6-monoesters of sucrose and then treating the ester mixture with a base to convert the sucrose 4-ester into sucrose 6-ester.

6. A process of claim 1 for the preparation of a sucrose 6-ester comprising steps of reacting sucrose in an inert organic solvent with a trialkyl orthoester or a ketene acetal in the presence of an acid catalyst to provide a sucrose alkyl 4,6-orthoester, which is further used as a raw material preparation of sucrose-6-acetate.

7. A process of claim 1 wherein pyridine is used as a solvent in extraction steps in production of TGS.
